(19) United States
(12) Patent Application Publication
(10) Pub. No.: US 2007/0244421 A1
(43) Pub. Date: Oct. 18, 2007

(54) Novel Occlusive Dressing

(75) Inventor: Neil William Graham, Glasgow (GB)

Correspondence Address: WORKMAN NYDEGGER, 60 EAST SOUTH TEMPLE, 1000 EAGLE GATE TOWER, SALT LAKE CITY, UT, 84111, US

(73) Assignee: THE SCOTTISH AMBULANCE SERVICE, Glasgow (GB)

(21) Appl. No.: 10/575,414

(22) PCT Filed: Oct. 8, 2004
(86) PCT No.: PCT/GB04/04265

§ 371 Date: Jul. 10, 2007

(30) Foreign Application Priority Data
Oct. 11, 2003 (GB) 0323881.3

(51) Int. Cl. A61F 13/00

(52) U.S. Cl. 602/43

(57) ABSTRACT

A novel occlusive dressing which allows the escape of fluid and air in cases of penetrating thoracic trauma. The dressing comprises a one-way valve that lies flat on the body of the patient during use such that the fluid from the wound is able to drain away without air from the surrounding environment penetrating the wound.

The present invention relates to the field of occlusive dressings and, more specifically, to occlusive dressings which allow the escape of fluid and air in cases of penetrating thoracic trauma.

In the majority of cases where serious injury occurs, it is necessary to apply a dressing to the wound in question. In the cases of penetrating thoracic trauma, an object like a bullet, knife or metal fragment for example has penetrated the chest wall, or both the chest wall and the lung itself, exposing the pleural space to the atmospheric pressure of the outside environment.

In order for a patient to breathe, it is necessary to maintain a proper pressure differential between the pleural cavity and the outside environment. Normally, the visceral and parietal pleura are separated by the pleural space, which itself is filled with pleural fluid. Typically, during inspiration, the rib cage expands, pulling the parietal pleura away from the visceral pleura. A negative pressure in the pleural space then develops in the lungs themselves, and positive atmospheric pressure forces air into the lungs.

When the normal negative pressure of the pleural space is exposed to the more positive atmospheric pressure in cases of trauma, outside air flows into the pleural space through the wound or through the punctured lung, causing a tension pneumothorax to develop. This is a very serious medical problem and eventually can result in the collapse of the lung, cardio-pulmonary collapse, unconsciousness, followed soon thereafter by cardio-pulmonary arrest and death.

It can therefore be seen that it is very important that a dressing be applied to the wound which prevents any air from entering the pleural space through the wound. However, at the same time, the dressing must allow air entering the pleural space through a punctured lung to escape from the wound, along with any blood that may have leaked into the pleural space as a result of the trauma. A dressing of this type would allow the proper pressure differential to at least partially be maintained between the pleural cavity and the outside environment.

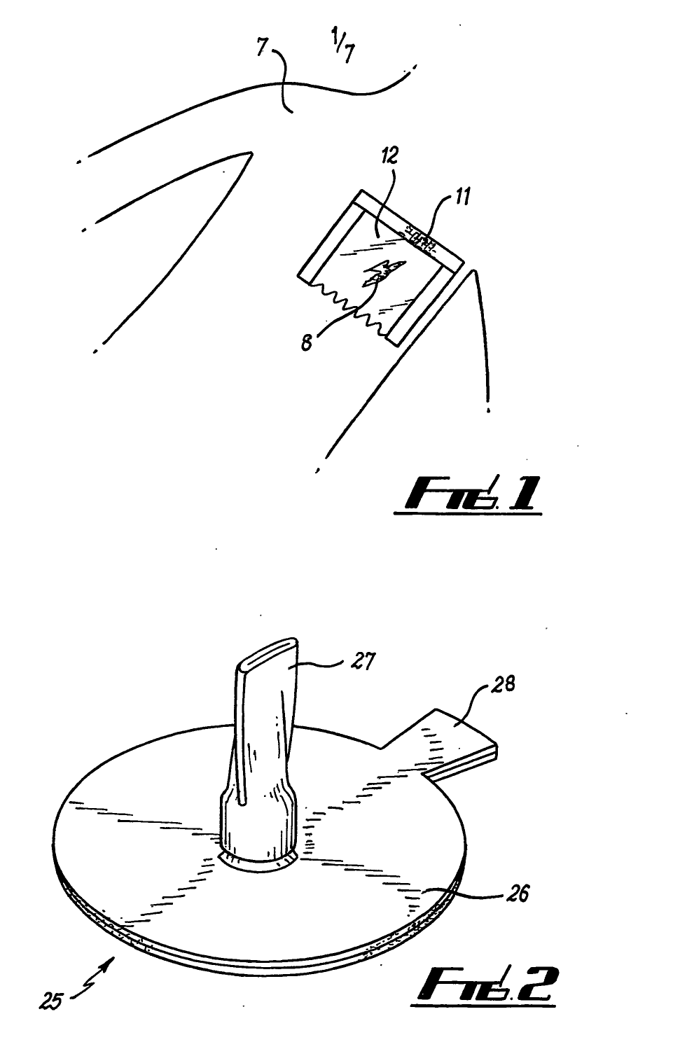

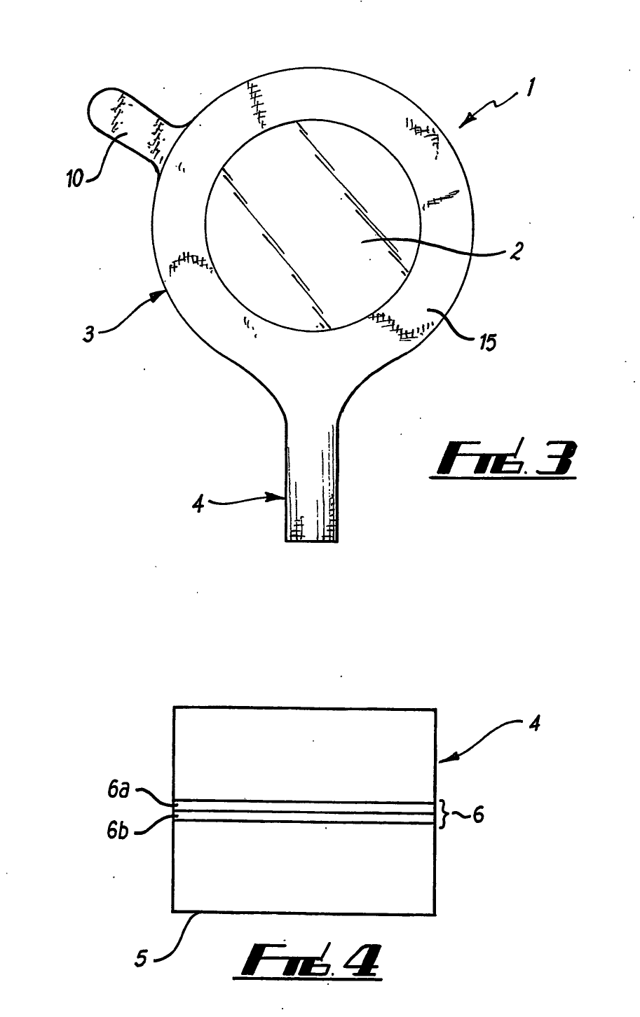

A number of dressings, solutions exist and have been used to try and deal with this issue. The most basic solution paramedics commonly use are ad-hoc dressings created on-site, an example of which is shown in FIG. 1, consisting of a piece of sterile plastic 12 laid flat on the patient 7 and wrap taped on three sides around the area of the wound 8 with adhesive tape 11. The fourth side is left open to allow air and fluid to escape. However, this is an extremely time consuming operation, and often results in the dressing being applied incorrectly, which can still lead to development of a tension pneumothorax. Other dressings also exist. Another example of a dressing 25 is shown in FIG. 2, and includes a planar surface 26 which adheres to the patient around the wound, the dressing also has a valve 26 protruding at 90° from the surface of the dressing. The dressing is also provided with a large grip tag 28. The dressing is opaque. This opacity results in the application of the dressing being difficult. The dressing design also can cause problems for the person applying the dressing to a patient, as discharges of bodily fluids are directed towards them. There is also the issue that as the valve 26 protrudes from the dressing 25, it is difficult to apply a number of dressings to, for example, both the front and rear of a patient, as in gun shot cases where there is both an entry and exit wound. There is also the issue that even in cases where such dressings are transparent to allow visualization of the wound, because the valve is protruding from the centre of the dressing, this obstructs the view of the wound.

It can be seen that it would be beneficial to be able to provide an occlusive dressing appropriate for use in penetrating thoracic trauma cases.

According to the present invention, there is provided a dressing comprising a bandage section, having a perimeter sufficient to occlude a wound, and a valve section that allows the escape of fluid, wherein the valve section lies substantially on the same plane as the bandage section.

Preferably the bandage section comprises a transparent area.

Preferably an outer edge of the bandage section is provided with adhesive.

Preferably the adhesive is covered with a removable protective outer film.

Preferably the valve section comprises a one-way valve.

Preferably the valve section comprises a flutter valve.

Optionally the flutter valve comprises one leaflet.

Alternatively the flutter valve comprises two leaflets.

Preferably the valve section further comprises a rigid casing.

Most preferably the one-way valve is housed in a substantially rigid casing.

Preferably the bandage section is substantially elliptical or circular in shape.

Preferably the bandage section is provided with an extending tag section allow for easy gripping.

Preferably the valve section can be provided with a collection bag.

Preferably the dressing is manufactured from a waterproof material.

Preferably the dressing is manufactured from non-allergenic material(s).

Preferably the dressing is manufactured from a plastic material.

Optionally the dressing is manufactured from latex.

Optionally the valve section is provided with an irrigation flushing system.

Preferably the irrigation flushing system comprises an aperture in the casing of the valve section through which fluid can be inserted.

Alternatively the irrigation flushing system comprises a tube and a valve.

Most preferably the tube is provided with perforations.

Optionally the bandage section comprises one or more membrane layers.

Preferably air or fluid is provided between the layers, such that tension is applied to a wound.

In order to provide a better understanding of the present invention, embodiments will now be described by way of example only and with reference to the following drawings, in which:

FIG. 1 shows an ad-hoc dressing used in emergency situations;

FIG. 2 shows prior art of an existing occlusive dressing; and

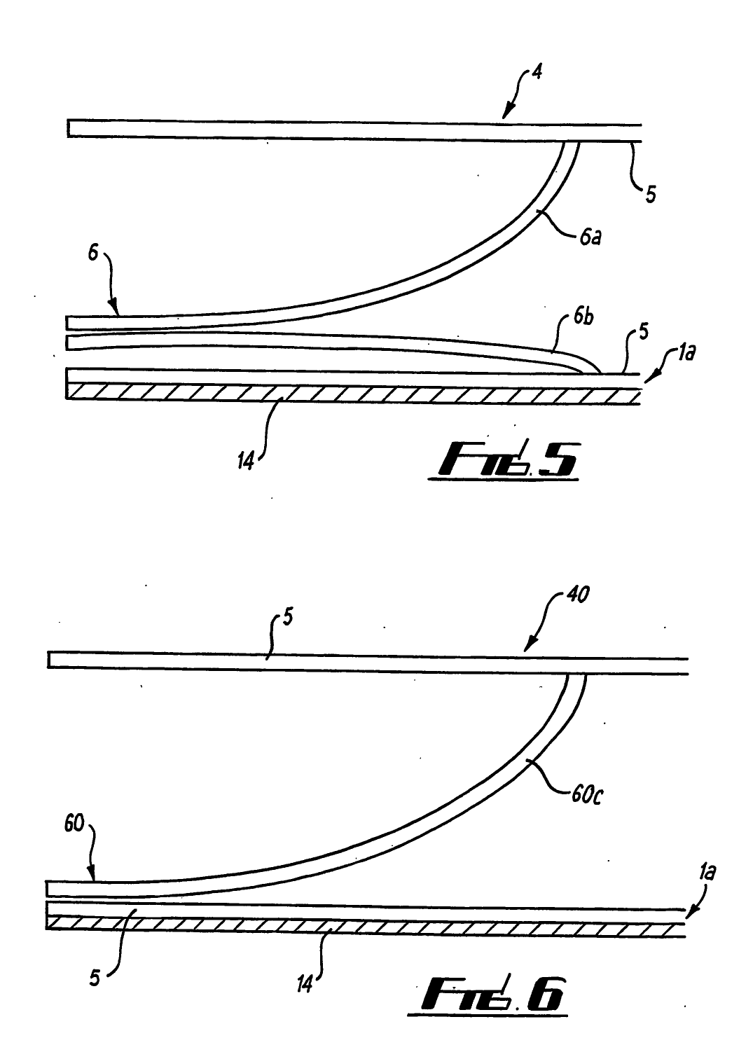

FIG. 3 shows a plan view of a dressing according to one embodiment of the present invention;

FIG. 4 shows a cross-section view of the valve section of the dressing of FIG. 3;

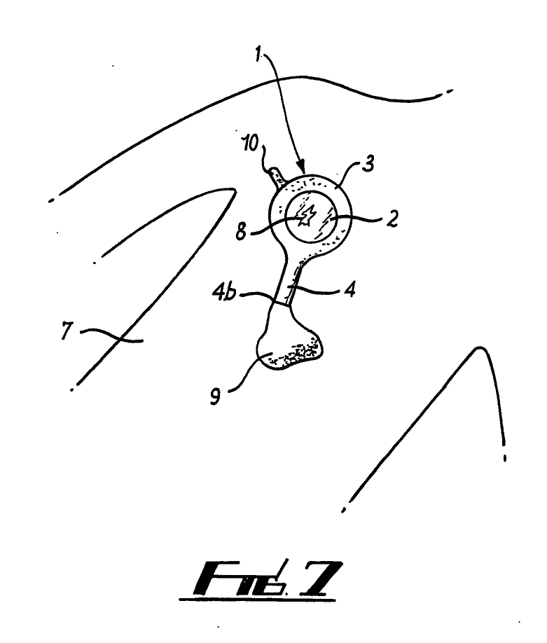

FIG. 5 shows a side cross-section view of the valve of FIG. 4;

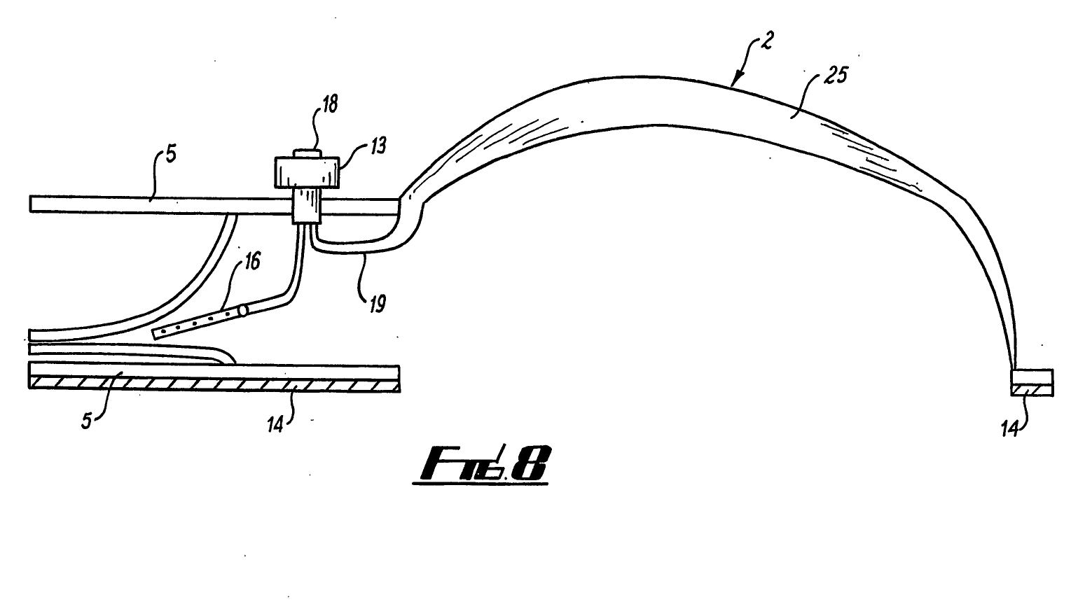

FIG. 6 shows an alternative embodiment of a valve according to the present invention;

FIG. 7 shows an example of the dressing in use according to the present invention;

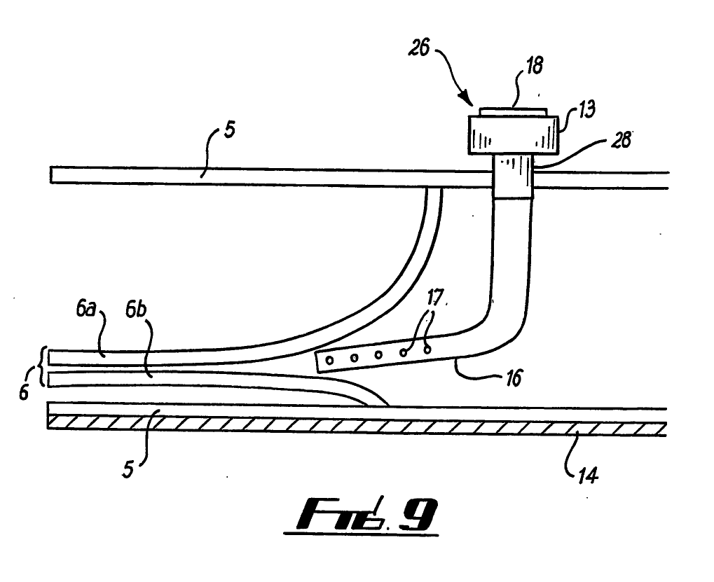

FIG. 8 shows a cross-section of a second embodiment of the dressing according to the present invention;

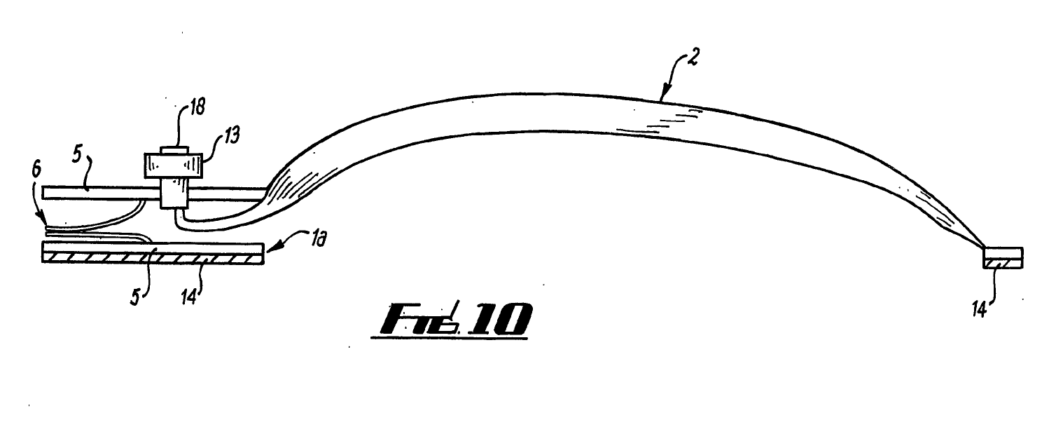

FIG. 9 shows a cross-section of a third embodiment of the valve section according to the present invention; and

FIG. 10 shows a cross-section of a fourth embodiment of the dressing according to the present invention.

As can be seen in FIG. 3, there is provided an occlusive dressing 1, the dressing having a bandage section 3 and a valve section 4. The bandage section 3 is substantially elliptical in shape, which provides it with ergonomic characteristics, making it easier to use than the standard square type bandages The bandage section 3 is provided with an outer area 15 and a central transparent area 2, which allows the underlying wound to be visualized by a paramedic or doctor, even whilst the dressing 1 is in use. The outer area 15 is coated on the underside with adhesive to ensure the dressing 1 adheres to the patient. Valve section 4 includes a one-way valve which allows the exiting of air and fluids from the wound over which the dressing is placed, whilst preventing the intake of air into the wound, which would disrupt the equilibrium in the pleural cavity. In this case the valve of the valve section 4 is a flutter valve.

The dressing 1 is often provided with a large grip tag 10, which allows both easy removal of the dressing 1 when required, even whilst the person providing assistance is wearing gloves. The tag also allows the adhesive on the reverse of the dressing 1 to be uncovered easily, as typically it will be covered in an appropriate material, such as thin plastic film, prior to use, which is then quickly removed when the dressing is required to be fixed in place on a patient.

FIG. 4 is a cross-section diagram of the valve section 4 of FIG. 3. Here it can be seen that in this embodiment the valve section 4 includes a flutter valve 6, wherein valve leaflets 6a and 6b are maintained together by atmospheric pressure, but allow the escape of fluids when pressure inside the dressing increases, but not the ingression of air. The flutter valve 6 is provided with a rigid outer casing 5, which strongly reduces the likelihood of the valve suffering blockages due to compression, making the valve section 4 generally more robust for use in emergency situations.

A side cross-section of the valve section 4 can be seen in FIG. 5. The valve section 4 has a rigid outer casing 5. The valve 6 is formed of an upper leaflet 6a and a lower leaflet 6b. The valve 6 allows fluids to escape from the site of the wound without allowing air, fluids or other matter entering the wound, as atmospheric pressure maintains the leaflets 6a and 6b in a closed position, however fluid can force its way through the valve 6 from within the dressing. The lower surface of the dressing 1a is provided with adhesive 14 to secure the dressing to the patient.

A cross-section of an alternative valve section 40 is illustrated in FIG. 6. In this arrangement the valve 60 comprises a single leaflet 60c which is maintained against the casing 5 by atmospheric pressure to act as a flutter valve.

FIG. 7 shows an example of the dressing 1 in use on a wound 8 position on the upper torso of a patient 7. It can be seen that the dressing 1 is positioned over the wound 8 with the ergonomic shape of the bandage section 3 and the transparent area 2, ensuring that the positioning of the dressing is both rapid and easy. The adhesive applied to the lower surface la of the outer area 15 and casing 5 secures the dressing 1 to the patient 7 providing an airtight seal around the wound 8 and thus allowing the dressing 1 to operate. Any air that is escaping from the wound 8, or any blood or liquid that is escaping due to the wound 8, is able to drain away through the valve section 4 on the dressing 1. However, as the valve 6 is a one-way valve, no air or liquid, etc., is able to regress back into the wound 8 from the external environment. In the illustrated embodiment, a collection bag 9 is positioned at the emission end 4b of the valve section 4 to allow air and bodily fluids to be kept in one place. This collection bag 9 can further be provided with apertures or will be produced in the matter that allows gaseous exchange with the external environment, so that liquid will be retained in the bag whilst gases emitted from the wound can escape.

The dressing 1 is designed in such a manner that it user friendly and can be manufactured in non-allergenic material, which increases the likelihood of adoption by medical and NHS staff. There are a number of benefits to this dressing 1 over and above dressings that have been suggested in the past. The ergonomic design of the dressing 1, along with the transparent nature of area 2 that is not obstructed by the valve section 4 in any manner, increases the speed of application and reduces the time that it would take to deliver a patient to hospital, for example.

The described dressing 1 also does not require careful positioning on the patient in order to allow the wound 8 to breathe, as the wound will be clearly visible. As covering a wound 8 and stopping it from breathing can lead to tension pneumothorax, it is advantageous that even a speedy application of the dressing 1 described in this invention would not cause this problem.

Another benefit to the dressing 1 described in the present invention is that the valve section 4 lies flat on the patient, as it is substantially on the same plane as the bandage section 3, rather than protruding perpendicularly, as in the case of previously described dressings. This protects the person applying the dressing 1 from the discharge of bodily fluids from the wound which offers improved health and safety conditions. The positioning of the valve section 4 also has the major benefit of allowing dressings to be applied to both the rear of the patient and the front of the patient, without resulting in discomfort or additional damage to the patient.

The transparent area 2 offers the applicator an unobstructed view of the wound 8, which is significantly beneficial over prior dressings, as it allows the applicator to monitor the condition of the wound 8, whilst still sealing the wound 8 and stopping air from penetrating, resulting in a sucking wound and the possibility of a tension pneumothorax. It also allows the person applying the dressing 1 to visualize the wound 8. The one-way valve reduces the possibility of infection or infectious agents penetrating the wound 8.

The dressing 1 described in the present invention can also be used as a pressure dressing to stem the flow of blood and improve the patient's situation. A cross-sectional diagram of such a dressing is shown in FIG. 8. As can be seen, the dressing 1 is provided with a membrane 19 which forms an additional layer 25 in the cavity of the dressing. The layer can be filled with air or fluid to provide constant tension on the wound acting to stem the flow of blood.

It is also worth noting that the use of a dressing 1, highlights the location and the possible type of wound 8 to the staff at a receiving hospital, allowing faster assessment of the wound 8.

The rigidity of the valve outer casing 5 is also very important, as it reduces the likelihood of valve blockages due to compression, i.e., by the patient rolling his body weight onto the dressing, and decreases the likelihood of a tension pneumothorax occurring. Such rigidity also allows the dressing 1 to be handled much more roughly, standing up to the extreme conditions often faced in an emergency situation.

FIG. 9 shows a cross-section of a third embodiment of the occlusive dressing which includes an irrigation flushing system 26. As can be seen, the valve section 4 is made up of an outer casing 5, as well the valve 6 comprising leaflets 6a and 6b. The outer casing 5 is formed of a rigid material such as hard plastic. An aperture 28 is provided in the outer casing 5 in which an inlet valve component 15 of the irrigation system 26 is positioned. Within the valve section 4, the irrigation system 26 comprises a tube 16, connected to the inlet valve 15. The tube is provided with perforations 17. The inlet valve may be a one-way valve, however it may alternatively be a rotating valve, a locking valve or any other type of appropriate valve. A syringe or similar fluid filled receptacle can be inserted into the aperture 18 of valve 15 and water or other appropriate fluid can be flushed through the valve section 4 to clean out the inside of the valve 6.

FIG. 10 shows the cross-section of a fourth embodiment of the dressing 1 which includes a combined irrigation and inflation function, the operation of which is controlled via the inlet valve 15, which in this case is a three-way valve.

The collection bag 9 may be removably attached to the base of the valve section 4, to allow the collection of any fluids, or it may be formed integrally with the dressing 1.

In conclusion, the dressing described in the present invention has a number of benefits over the prior art. However, the abovementioned description should not be taken as being limiting, as further modifications and improvements can be made by one skilled in the art within the scope of the invention herein disclosed.

It should be noted that the lower surface 1a may be coated directly with adhesive, however it may alternatively be provided with a layer of closed cell foam which is coated with adhesive. The closed cell foam ensuring the dressing confirms to the contour of the patient's skin and providing a complete seal.

Although the illustrated dressing is provided with a tag, however this tag is provided merely to improve ease of handling, the dressing does not need to be provided with a tag.

Ideally the dressing is transparent, but the dressing is still functional and will help manage the condition of the patient even if not transparent.

The dressing should be waterproof material, ideally it can be formed of plastics.

1. A dressing comprising
a bandage section having a perimeter sufficient to occlude a wound, and
a valve section that allows the escape of fluid, wherein the valve section lies substantially on the same plane as the bandage section.

2. A dressing, as in claim 1, wherein the bandage section comprises a transparent area.

3. A dressing, as in claim 1, wherein an outer edge of the bandage section is provided with adhesive.

4. A dressing, as in claim 3, wherein the adhesive is covered with a removable protective outer film prior to use.

5. A dressing, as in claim 1, wherein the valve section comprises a one-way valve.

6. A dressing, as in claim 5, wherein the one-way valve comprises a flutter valve.

7. A dressing, as in claim 6, wherein the flutter valve comprises one leaflet.

8. A dressing, as in claim 6, wherein the flutter valve comprises two leaflets.

9. A dressing, as in claim 5, wherein the one-way valve is housed in a substantially rigid casing.

10. A dressing, as in claim 1, wherein the bandage section is substantially elliptical or circular in shape.

11. A dressing, as in claim 1, wherein the bandage section is provided with an extending tag section, wherein the extending tag section facilitates easy gripping of the dressing.

12. A dressing, as in claim 1, wherein the valve section can be provided with a collection bag.

13. A dressing, as in claim 1, wherein the dressing comprises a waterproof material.

14. A dressing, as in claim 1, wherein the dressing comprises a non-allergenic material(s).

15. A dressing, as in claim 1, wherein the dressing comprises a plastic material.

16. A dressing, as in claim 1, wherein the dressing comprises a latex.

17. A dressing, as in claim 1, wherein the valve section is provided with an irrigation flushing system.

18. A dressing, as in claim 17, wherein the irrigation flushing system comprises an aperture in the casing of the valve section through which fluid can be inserted.

19. A dressing, as in claim 17, wherein the irrigation flushing system comprises a tube and a valve.

20. A dressing, as in claim 19, wherein the tube is provided with perforations.

21. A dressing, as in claim 1, wherein the bandage section comprises one or more membrane layers.

22. A dressing, as in claim 21, wherein the bandage section comprises two or more membrane layers, and wherein, in use, air or fluid is provided between the membrane layers, such that tension is applied to a wound.
